(19) United States
(12) Patent Application Publication
(10) Pub. No.: US 2013/0085567 A1
(43) Pub. Date: Apr. 4, 2013

(54) Method and apparatus for performing DMEK surgery

(71) Applicants: Donald Tan, Singapore (SG); Jodhbir Mehta, Singapore (SG)

(72) Inventors: Donald Tan, Singapore (SG); Jodhbir Mehta, Singapore (SG)

(21) Appl. No.: 13/573,661

(22) Filed: Oct. 1, 2012

Related U.S. Application Data
(60) Provisional application No. 61/541,431, filed on Sep. 30, 2011.

(51) Int. Cl. A61F 2/14

(52) U.S. Cl. 623/5.12

(57) ABSTRACT

DMEK surgery is performed using a carrier mat made of a polymeric material having elastic memory and a hydrophilic surface, for supporting the descemet's membrane and transporting it to an insertion capsule.

### CROSS-REFERENCE TO RELATED APPLICATION

This application claims the benefit of U.S. Provisional patent Application No. 61/541,431, filed Sep. 30, 2011, the entire specification of which is incorporated hereinto by reference.

### BACKGROUND OF THE INVENTION

Corneal transplantation (penetrating keratoplasty (PK)) was first performed by Zirm over 104 years ago. Until recently there have been few changes compared to the original surgery, however over the last decade there have been dramatic improvements in surgical techniques (1). There are many complications related to conventional penetrating keratoplasty e.g. poor ocular surface healing, neutrophic cornea, suture related complications, graft rejection, tectonically weak eyes and high graft astigmatism hence surgeons have been looking at more selective tissue lamellar transplantation techniques to circumvent some of these problems.

The cornea is comprised of essentially 3 main layers, the epithelium, the stroma and the descemet's membrane (DM)/endothelial cell complex. Selective tissue transplantation procedures have taken the form of either anterior lamellar keratoplasty (ALK) in which the epithelium and stroma are replaced or endothelial keratoplasty (EK) in which essentially the DM/endothelial cell complex is replaced, with or without a posterior stromal carrier for support. In patients with purely corneal endothelial dysfunction, selective tissue transplantation, in the form of endothelial keratoplasty, has emerged as a viable alternative to full thickness conventional penetrating keratoplasty. The 2010 EBAA (Eye Banks Association of America) Statistical Report revealed that of the 42, 642 corneal transplants performed in the US, 19,159 were EK procedures, representing 44.9% of all transplants, a 9.5% increase compared to 2009. In Europe, EK is also becoming a mainstream procedure (constituting 28% of all grafts in the UK), whereas Asia has been slower to adopt EK with Singapore being an exception—in 2010, 36% of all transplants in Singapore were EK procedures. Endothelial dysfunction is the leading cause of corneal transplantation in Singapore and USA, as well as in many European countries such as the UK and Sweden.

Endothelial keratoplasty was first described by Melles in 1998. The surgery involves manual dissection of both the donor and the recipient cornea and transplantation of essentially the donor posterior stroma with DM and endothelium (Deep lamellar endothelial keraoplasty (DLEK)). The surgery requires special instrumentation but obviated the need for surface sutures as in PK. Hence a proof of concept was established. Due to the interface between the donor and recipient stroma, haze was often noted at the interface and this led to a reduction in best spectacle-corrected visual acuity. However the results were equivalent to those achieved with PK surgery with respect to visual acuity outcomes but the surgery often took many hours. To combat the problem of the interface and the manipulation in the recipient cornea, Melles described a technique where the recipient endothelium was simply stripped off in a technique called descemet's stripping endothelial keratoplasty (DSEK). This removed the need for dissection of the recipient cornea and reduced surgical time dramatically. However, the donor cornea was still transplanted as a 150-250 micron thick donor lenticule comprising of posterior stroma DM/endothelium. Since there was minimal dissection of the recipient cornea, the donor lenticule effectively protruded from the posterior edge of the cornea into the anterior chamber. Price et al further improved the surgery by involving the use of an automated machine to cut the donor lenticule. This provided a smoother stromal interface of the donor lenticule and allowed for a more consistent thickness of the donor lenticule, compared to the original manual dissection technique (DSEK). This surgery was coined DSAEK (Descemet stripping automated endothelial keratoplasty).

DSAEK has become synonymous with endothelial transplantation and is currently the dominant form of EK worldwide, accounting for well over 90% of all EK procedures, and results have been shown to be equivalent and in some centres superior to penetrating keratoplasty, and recently has been shown to have a significantly lower rate of transplant rejection as compared to PK. Essentially it is sutureless, small incision corneal transplantation surgery. There are no sutures on the ocular surface and the posterior cornea is accessed by a 5 mm scleral incision. The recipient DM/diseased endothelium is stripped off and the donor cornea is inserted into the anterior chamber of the recipient. The new graft is then positioned against the recipient posterior stroma using air tamponade for 8 min. Following which there is air fluid exchange, leaving the anterior chamber with an 75% air fill overnight.

Even though visual acuity results and outcomes have been highly encouraging with this surgery, the operation is not an exact anatomical tissue replacement procedure. The donor cornea is made up of stroma DM/endothelium hence it is not an exact tissue replacement since the recipient is prepared by stripping the diseased DM/endothelium. This may compromise the donor due to the extra thickness in eyes with shallow anterior chamber or with anterior chamber intraocular lenses, and anterior chamber comprise with peripheral anterior synechiae is a well known complication. Also it adds a potential second interface between the donor stroma and the recipient which may further degrade visual quality.

sDMEK (Descemets Membrane Endothelial Keratoplasty

Melles in 2006, subsequently described a technique called descemet membrane endothelial keratoplasty (DMEK). In this technique just the DM/endothelium was harvested from a donor and then transplanted into a recipient in which DM/endothelium was stripped. DMEK potentially offers major advantages over the earlier three techniques. Firstly, visual rehabilitation is much faster. Secondly, a near perfect anatomical restoration of the recipient cornea in DMEK has clearly been proven to provide better optical quality of the recipient cornea, with a significantly higher percentage of patients attaining perfect (6/6) best correction visual acuity, and thirdly, in contrast to DSAEK in which the donor is prepared by automated therapeutic lamellar keratoplasty unit to produce a 100-250 microns thick tissue, the donor DM/endothelium can be peeled off directly in DMEK, and hence does not require the need for additional expensive surgical equipment. Also the transplantation of only the DM/endothelium has led to reportedly reduced rates of graft rejection, even surpassing that of DSAEK. A recent article currently in press by Price et al showed that rejection rates in PK, DSAEK and DMEK in Fuchs' and pseudophakic bullous keratoplasty were 17%, 9% and 0.7% respectively.

The major challenge of DMEK is three-fold:

(1) the harvesting of the DM/endothelial complex without excessive endothelial cell damage;

(2) the difficulty in handling the 10 micron DM sheet, which tends to ‘roll up’ due to its thickness (only 10 microns). Since the roll develops with the endothelial cells on the outside there is further damage to the endothelium when the “Descemet-roll” is unrolled in the eye before tamponading to the recipient cornea.

(3) unscrolling the DM roll in the anterior chamber to allow attachment to the recipient.

Currently only a small handful of surgeons worldwide are performing DMEK on a routine basis, simply because of the surgical challenges described above. While donor preparation techniques have generally been solved, with several techniques described now enabling effective donor stripping with minimized tissue loss from tearing or endothelial damage, the major challenge in DMEK lies in handling the scrolled DM which always scrolls with the endothelial surface outwards, so that any handling results in endothelial cell damage. Tight scrolling is often encountered in young donors below the age of 40 years, and hence only older donors over the age of 50 years are generally used in DMEK surgery (another reason to use older donors is that DM stripping of the donor is also more difficult in younger donors). Insertion of this scroll of DM is relatively simple to achieve through a small corneal or scleral incision, usually be placing the scroll into a IOL or Phakic IOL cartridge injector and injecting the scroll into the AC, but he main surgical challenge lies in unscrolling the donor into the right endothelium-down orientation in the AC, and aligning the donor centrally, and without wrinkling onto the posterior surface of the recipient cornea.

This is achieved by injecting short bursts of BSS with a cannula through several paracentesis ports to initiate unscrolling, injecting various sizes of air bubbles beneath the donor to further unscroll the tissue, and finally injecting the AC with a full chamber of air to achieve tamponade and adherence of the donor to the recipient cornea. This process may take anywhere from 10 minutes to an hour to perform, and as a results, currently, only 3 main surgeons (Melles in Rotterdam, Price in the US, and Kruse in Germany) have reported in reasonable series of DMEK cases. Despite this, their studies collectively report much better visual results compared to DSEK, but with a higher rate of donor rebubbling due to residual donor detachment, and endothelial cell loss rates which are higher, or just able to match DSEK cell loss rates. Many corneal surgeons today feel that DMEK will never become a mainstream procedure unless the surgery becomes technically easier and more predictable.

### BRIEF SUMMARY OF THE INVENTION

It is the broad objects of the present invention to provide a novel method and system for aiding manipulation of the thin DM/endothelial complex, so as to greatly simplify the donor handling, and AC manipulation stages, of DMEK surgery.

It has now been found that certain of the foregoing and related objects of the invention are attained by the provision of a method for performing DMEK surgery, wherein the improvement comprises:

providing a thin, flexible, self-supporting, generally circular carrier mat having elastic memory and an hydrophilic surface;

inserting the carrier mat under a descemet's membrane separated from a donor stroma with the endothelial surface on the membrane upwardly disposed and out of contact with the carrier mat, and with the hydrophilic surface of the carrier mat in contact with the surface of the membrane opposite to the endothelial surface;

centering the descemet's membrane on the carrier mat with an annular marginal portion of the carrier mat surrounding the membrane and extending therebeyond, and causing the membrane to lie in a flat, wrinkle-free condition on the carrier mat;

providing a glide capsule that is constructed to form the assembled carrier mat and carried membrane into a double-coiled scrolled configuration upon being drawn thereinto, and for introducing the membrane assembly, in double-coiled scrolled configuration, into the anterior chamber of a recipient's eye;

drawing the carrier mat and membrane assembly into the glide capsule to form the assembly into a double-coiled scrolled configuration with the endothelial surface of the membrane facing inwardly and with no area of epithelial cells in substantial contact with any other such area thereof;

using the glide capsule to transfer the membrane, in double-coiled scrolled configuration and proper orientation, into the anterior chamber of a recipient's eye through an incision on one side of the anterior chamber by initially partially inserting the glide capsule into the incision on the one side of the anterior chamber;

grasping a marginal portion of carrier mat, using an instrument inserted through an incision on the opposite side of anterior chamber, and withdrawing the carrier mat partially from glide capsule;

drawing the descemet's membrane away from the carrier mat, by force applied to the membrane using an instrument inserted through the opposite-side incision, and into the anterior chamber;

withdrawing the glide capsule from the incision on the one side of the anterior chamber while maintaining restraining force on the descemet's membrane, as necessary, and together with the glide capsule withdrawing the carrier mat, partially retained in the glide capsule, from the anterior chamber, leaving the membrane within the anterior chamber;

causing the descemet's membrane to unscroll within the anterior chamber, the proper orientation being such that the endothelial surface is downwardly oriented and the carrier mat being fabricated from a polymer that is sufficiently flexible and soft, and that has sufficient tensile strength and resistance to tearing, to permit it to perform the carrier functions, and to withstand the manipulations required, in carrying out the method.

Normally, the carrier mat will be about 30 to 100 millimeters thick; the surrounding annular portion, extending beyond the descemet's membrane, will be about 0.5 millimeter in radial width, with the diameter of the descemet's membrane being about 8 to 9 millimeters; the carrier mat will have an inherently arcuate cross section, with the hydrophilic surface thereof inwardly disposed; and the polymer from which the carrier mat is fabricated will be a silicone hydrogel.

Other objects of the invention are attained by the provision of a system for performing DMEK surgery comprising, in combination: a thin, flexible, self-supporting circular carrier mat having an elastic memory and an hydrophilic surface; and an inserter comprised of glide capsule having open opposite ends and an interior chamber, and being constructed to receive the carrier mat through one of the open ends and to form said mat into a double-coiled scrolled configuration upon being drawn into the interior chamber thereof. The carrier mat will normally have the features hereinabove and hereinafter disclosed. The guide capsule will preferably be constructed forwardly tapered conical portion, terminating in an open end, and will be constructed to provide a ridge that extends between the open ends for assisting in forming the carrier mat into such a double-coiled scrolled configuration.

### BRIEF DESCRIPTION OF THE SEVERAL VIEWS OF THE DRAWING

FIG. 1 is a plan view of a DM support and transfer, or carrier, mat suitable for use in the practice of the present invention;

FIG. 2 is a side elevational view of the carrier mat;

FIG. 3 is a plan view of a DM/carrier mat complex, or assembly;

FIG. 4 is a fragmentary plan view of a glide capsule and introducer suitable for use in the practice of the invention;

FIG. 5 is an end view of DM/carrier mat complex in a coiled configuration produced within a glide capsule, with the endothelial cell surface of the DM facing inwardly and with no area of the endothelial cell surface contracting any other such area thereof;

FIG. 6 is an elevational view of forceps suitable for use in the practice of the present invention; and

FIG. 7 is an elevational view of the forceps of FIG. 6, wherein a head component is detached from the handle.

### DETAILED DESCRIPTION OF THE INVENTION

### Product 1: Supporting and Transfer Mat

The supporting and transfer, or carrier disc or mat, employed in the practice of the present invention, sometimes referred to by the trade designation “DMat,” is a device that affords structural rigidity to the thin DM/endothelial complex to:

a) prevent inadvertent scrolling;

b) prevent wrinkling;

c) prevent inadvertent eversion of the donor;

d) allow for handling of the donor using the carrier mat, thus reducing endothelial damage; and

e) facilitate donor entry and manipulation in the AC.

### Description of the Carrier Mat

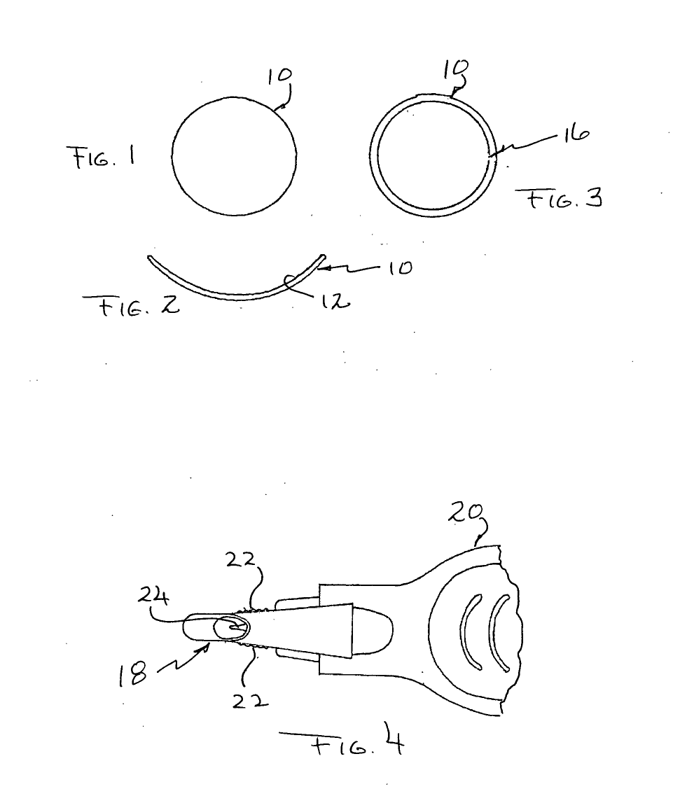

The Carrier Mat, generally designated by the numeral 10 in FIGS. 1 through 3 and 5, is a disposable, single-use, circular, transparent mat approximately 30-100 microns thick and may be composed of a variety of non-dye absorbent polymer materials, which are highly flexible and soft, but has good tensile strength, resistance to tearing, and elastic memory so as to enable coding when drawn into a chamber and natural uncoiling when removed therefrom. The mat 10 has an hydrophilic surface 12 with low surface tension (normally, of course, the carrier mat will be a made from a single polymer, with both surfaces inherently having those properties), and a curvature that matches any standard corneal suction trephination block. The Carrier Mat may be clear or be slightly colored to distinguish this from the DM complex, while aiding visualization of the Carrier Mat in the AC and when placed in the Glide Capsule. Although circular, it may have a 0.5 mm to 1.0 mm protruding tab (not shown) to facilitate grasping of the Carrier Mat with intraocular forceps (depicted in FIGS. 6 and 7).

While, as noted above, a preferred material for fabrication of the Carrier Mat is a silicon hydrogel, other suitable polymers will be apparent to those skilled in the art in view of the disclosure of the present specification. Additional information concerning suitable materials is provided in International Publication WO 2012/118681, the pertinent disclosure of which publication is incorporated hereinto by reference thereto.

### Product 2: DMEK Inserter Device

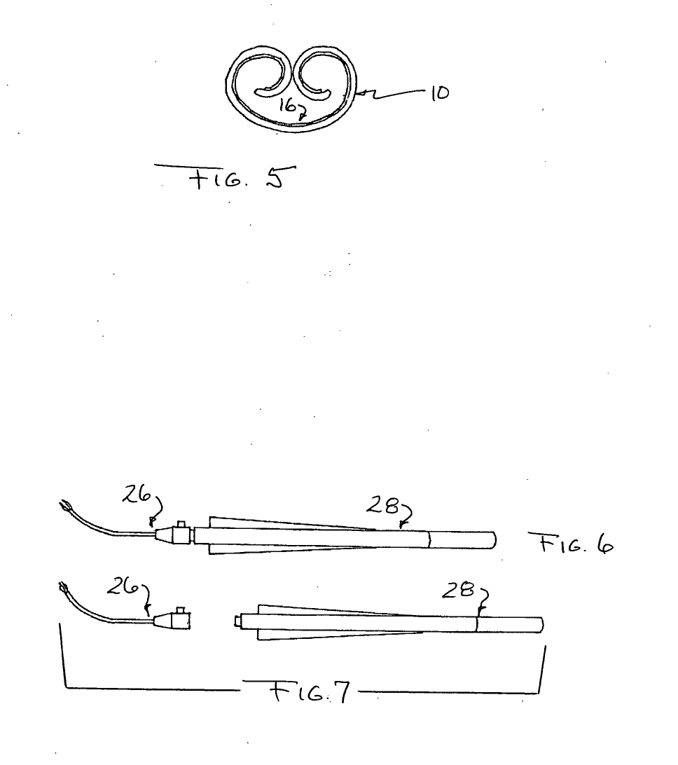

The DMEK Inserter, depicted in FIG. 4 hereof, is a glide pull-through device based on the original Tan EndoGlide, consisting of a Preparation Base (not shown), Glide Capsule, generally designated by the numeral 18, and Glide Introducer, generally designated by the numeral 20, all modified to match the thinness of the DM complex and customized to fit through a smaller corneal or scleral incision. Such a preparation base, glide capsule, and glide introducer (or insert member) are disclosed in Weston et al. U.S. patent application Ser. No. 13/374,293, published on Sep. 6, 2012 under number US-2012-0226286-A1, the disclosure of which is hereby incorporated hereinto by reference. FIGS. 12 and 17 of the Weston et al application are particularly germane, the latter showing the tapered conical Glide Capsule, which is (as seen in FIG. 4 hereof) smaller in diameter in front and larger in diameter posteriorly, so as to enable the smallest wound, which will be of trapezoidal configuration. The outer lateral sides of the Glide Capsule are ribbed, as seen at 22, or have multiple ridges to prevent expulsion of the device once it is fully introduced through the wound.

With regard to the internal diameter of the Glide Capsule 18, for a 9 millimeter DMEK donor on a 10 millimeter Carrier Mat, one may double coil this DM complex through a minimum diameter of 2.9 millimeters without a central ridge. With a central ridge 24 of 0.5 millimeter in length, it is further possible to utilize a 2.57 millimeter internal diameter without endothelial touch or overlap. As such, the minimum internal diameter of the Glide Capsule will be in the region of 2.60 to 3.00 millimeters. Accounting for the thickness of the walls, the Glide Capsule 18 will be tapered such that the leading or front portion will be approximately 2.7 to 3.0 mm, while the back portion will measure around 3.5 to 4.00 millimeters. The internal ridge 24 will prevent endothelial touch, as in the prior version, and the Capsule will be sealed by the Glide Introducer 20 to ensure a complete seal.

The well of the Preparation Base (not shown in the append drawings) will be more elevated compared to the original EndoGlide device so as to receive the Carrier Mat/DM complex without placement of an anterior lamellar cap.

Product 3: Detachable curved DMEK Intraocular Forceps and Accompanying Sponge Support

The Detachable curved DMEK Intraocular Forceps, shown in FIGS. 6 and 7, is suitable for insertion through incisions into the anterior eye chamber, for use in gripping and applying drawing and restraining force, and is similar to the current curved EndoGlide forceps but has a head, generally designated by the number 26, which can be detached from the main handle of the forceps, generally designated by the number 28, while it is in place through the paracentesis and while still grasping the DM complex. The detachable head has a clip attached to open the teeth of the forceps independently of the handle. To support the head and leading cannula of the forceps while in the AC, a foam or sponge support (not shown) is used to support the head of the forceps and maintain the exact angulation of the cannula, with the foam support placed on the nasal canthal area.

DMEK Surgical Technique using the Carrier Mat and the DMEK Inserter Device
a) Donor Preparation: The DM is first peeled off the recipient cornea in a manner similarly to that is conventional but with a major difference, which is to effect donor peeling without immediate scrolling after complete separation from the donor stroma. This is performed under a small level of Optisol or BSS using a suction block in order to provide stabilization of the donor. The DM is first scored anterior to schwalbe line. This is then extended circumferentially. The DM is then carefully elevated using a blunt dissector and the dissection is extended both circumferentially and radially. Trypan Blue is added to the improve visualization of the DM complex to stain the DM complex a deep blue. Trephination of the donor to the desired diameter (usually ranging from 8.0 mm to 9.0 mm) is then performed. The complete separation of the trephined DM is then completed with the use of forceps to peel away the donor. It is crucial at this stage that not too much fluid in the suction block chamber is present, so that once the donor is fully separated, it is not allowed to scroll up due to the shallowness of the fluid in the suction block.
b) Donor placement on the Carrier Mat:

The curved Carrier Mat is then carefully slid under the DM while it is still lying flat in the donor block chamber, again ensuring that excessive fluid is not present to initiate scrolling, and that the endothelial surface is not touched during the sliding process. The Carrier Mat is sized to be 1 millimeter larger than the dissected DM, and thus would be available in a variety of diameters, or conversely can be trephined to the right size by the surgeon. Following placement of the DM on the Carrier Mat, careful centering of the DM on the Carrier Mat is performed to ensure that an equal 0.5 millimeter exposure of the Carrier Mat is achieved all around the DM, and that the DM lies completely flat and unwrinkled on the Carrier Mat. Gentle wicking away of excess fluid around the edge of the Carrier Mat and in the suction block chamber is the performed to enable the DM to fully contact the Carrier Mat with minimal intervening fluid, so that the DM now is moderately adherent to the Carrier Mat by capillary action.
c) Loading/coiling of the Carrier MatIDM complex into the DMEK Glide Chamber:

The Carrier Mat/DM complex is slid under a Paton spatula and placed onto the well of the Glide Capsule, with the Carrier Mat tab (if any) adjacent to the Glide Capsule opening.
OVD is placed on the endothelial surface, and the EndoGlide loading forceps is passed through the front end of the Glide Capsule, protruding out the back end, to grasp the periphery of the Carrier Mat. The Carrier Mat with the attached DM complex is then pulled into the Glide Capsule, with double coiling achieved with the central ridge 24, and aided by placing a BSS cannula vertically in alignment with the central ridge to ensure that the curling edges of the Carrier Mat do not overlap (see FIG. 5). The Carrier Mat is advanced through the Capsule so that it just appears from the anterior edge opening of the Capsule. The loaded Glide Capsule is then removed from the Preparation Base, and everted the right way up ready for insertion into the recipient eye.
d) Insertion of the DMEK Insertion Device into the wound and delivery of the DM complex into the AC:

The device is inserted into the AC through the 4.6-3.5 millimeter incision. Once fully inserted into the AC, and with an AC maintainer present on low flow so as to achieve a slightly shallow, but intact anterior chamber, DMEK intraocular forceps is introduced through a nasal paracentesis, and is used to grasp the tab of the Carrier Mat. The Carrier Mat is then gradually pulled halfway out of the Glide Capsule, into the AC, and is released. The same forceps then is used to grasp the leading edge of the DM complex and the DM complex is gently pulled away from the Carrier Mat, out of the Glide Capsule, and fully into the AC. While still holding onto the DM complex, the Glide Capsule is then carefully retracted out of the eye, and the Carrier Mat will also follow with the Glide Capsule, detaching itself completely from the DM complex. Once the Glide Capsule and Carrier Mat is fully out of the eye, a small air bubble is injected with a 30 G needle under the uncoiling DM complex.
e) Manipulation and positioning of the DM complex in the AC:

The DM complex is fully uncoiled and opened with the use of BSS and air bubble injection, while it is still held by the intraocular forceps. The handle of the intraocular forceps may be detached from the head and cannula of the forceps while still holding onto the DM complex, and the head and cannula is supported by the foam support which is placed under the head of the cannula, over the nasal canthal area. This enables the surgeon to let go of the forceps which is still holding the DM complex in place so that both hands are free to inject BSS and air and maneuver the DM complex into a fully uncoiled and central position just under the recipient cornea. Once the DM complex is perfectly positioned in relation to the recipient cornea, more air is injected to ensure stability of the DM com-plex, and finally the forceps hold on the DM complex can be released using the clip on the head of the forceps. The corneal or scleral wound is sutured, the AC maintainer removed and the wound sutured, and a complete air fill with air tamponade completes the DMEK procedure.

Having thus described the invention, what is claimed is:

1. In a method for performing DMEK surgery, the improvement comprising:
providing a thin, flexible, self-supporting, generally circular carrier mat having elastic memory and an hydrophilic surface;
inserting said carrier mat under a descemet's membrane separated from a donor stroma with the endothelial surface on said membrane upwardly disposed and out of contact with said carrier mat, and with said hydrophilic surface of said carrier mat in contact with the surface of said membrane opposite to said endothelial surface;
centering said descemet's membrane on said carrier mat with an annular marginal portion of said carrier mat surrounding said membrane and extending therebeyond, and causing said membrane to lie in a flat, wrinkle-free condition on said carrier mat;
providing a glide capsule that is constructed to form said assembled carrier mat and carried membrane into a double-coiled scrolled configuration upon being drawn thereinto, and for introducing said membrane assembly, in double-coiled scrolled configuration, into the anterior chamber of a recipient's eye;
drawing said carrier mat and membrane assembly into said glide capsule to form said assembly into a double-coiled scrolled configuration with said endothelial surface of said membrane facing inwardly and with no area of epithelial cells in substantial contact with any other such area thereof;
using said glide capsule to transfer said membrane, in double-coiled scrolled configuration and proper orientation, into the anterior chamber of a recipient's eye through an incision on one side of the anterior chamber by initially partially inserting said glide capsule into said incision on said one side of said anterior chamber;
grasping said marginal portion of said carrier mat, using an instrument inserted through an incision on the opposite side of said anterior chamber, and withdrawing said carrier mat partially from said glide capsule;
drawing said descemet's membrane away from said carrier mat, by force applied to said membrane using an instrument inserted through said opposite-side incision, and into said anterior chamber;
withdrawing said glide capsule from said incision on said one side of said anterior chamber while maintaining restraining force on said descemet's membrane, as necessary, and together with said glide capsule withdrawing the carrier mat, partially retained in said glide capsule, from said anterior chamber, leaving said membrane within said anterior chamber;
causing said descemet's membrane to unscroll within said anterior chamber, said proper orientation being such that said endothelial surface is downwardly oriented;
said carrier mat being fabricated from a polymer that is sufficiently flexible and soft, and that has sufficient tensile strength and resistance to tearing, to permit it to perform the carrier functions, and to withstand the manipulations required, in carrying out said method.

2. The method of claim 1 wherein said carrier mat is about 30 to 100 microns thick.

3. The method of claim 1 wherein said surrounding annular portion of said carrier mat, extending beyond said descemet membrane, is about 0.5 millimeter in radial width.

4. The method of claim 3 wherein the diameter of said donor descemet membrane is about 8 to 9 millimeters.

5. The method of claim 5 wherein said carrier mat is of inherently arcuate cross section, with said hydrophilic surface thereof inwardly disposed.

6. The method of claim 1 wherein said polymer is a silicone hydrogel.

7. In a system for performing DMEK surgery, the combination comprising:
a thin, flexible, self-supporting circular carrier mat having an elastic memory and an hydrophilic surface; and
an inserter comprised of glide capsule having open opposite ends and an interior chamber, and being constructed to receive said carrier mat through one of said open ends and to form said mat into a double-coiled scrolled configuration upon being drawn into said interior chamber thereof.

8. The system of claim 7 wherein said carrier mat is about 30 to 100 microns thick and about 9 to 10 millimeters in diameter.

9. The system of claim 7 wherein said carrier mat has sufficient tensile strength and resistance to tearing to permit it to be drawn, without damage, into said capsule and withdrawn therefrom.

10. The system of claim 7 wherein said carrier mat has an inherently acruate cross section.

11. The system of claim 7 wherein said carrier mat is fabricated from a silicone hydrogel polymer.

12. The system of claim 7 wherein said capsule is constructed with forwardly tapered conical portion terminating in an open end.

13. The system of claim 7 wherein said capsule has a ridge extending between said open ends for assisting in forming said carrier mat into such a double-coiled scrolled configuration.
